(19) United States
(12) Patent Application Publication
(10) Pub. No.: US 2020/0121960 A1
(43) Pub. Date: Apr. 23, 2020

(54) REVERSIBLE THERMAL NEUROMODULATION USING FOCUSED ULTRASOUND

(71) Applicant: REGENTS OF THE UNIVERSITY OF MINNESOTA, MINNEAPOLIS, MN (US)

(72) Inventors: David Darrow, Minneapolis, MN (US); Emad S. Ebbini, Edina, MN (US); Parker D. O'Brien, Minneapolis, MN (US); Theoden Netoff, Minneapolis, MN (US)

(21) Appl. No.: 16/586,357

(22) Filed: Sep. 27, 2019

Related U.S. Application Data
(60) Provisional application No. 62/738,420, filed on Sep. 28, 2018.

(51) Int. Cl. A61N 7/02

(52) U.S. Cl. CPC A61N 7/02; A61N 2007/0026

(57) ABSTRACT

A method includes positioning an ultrasound transducer system to direct focused ultrasound energy into a subject's tissue, configuring the ultrasound transducer system to direct focused ultrasound energy into target nervous system tissue in the subject's tissue configured to cause heating of the target nervous system tissue to reversibly modulate neural activity, and delivering focused ultrasound energy to target nervous system tissue based on the configured ultrasound transducer system. A system includes a dual-mode ultrasound transducer and a controller configured to deliver focused ultrasound energy.

The present application claims the benefit of U.S. Provisional Application No. 62/738,420, filed Sep. 28, 2018, which is incorporated by reference.

### GOVERNMENT FUNDING

This invention was made with government support under NS098781 awarded by the National Institutes of Health. The government has certain rights in the invention.

### FIELD

The present technology is generally related to neuromodulation. In particular, the present technology is related to reversible neuroinhibition using ultrasound.

### SUMMARY

This disclosure generally relates to low intensity focused ultrasound (LIFU) for thermal neuromodulation. Compared to existing noninvasive, neuromodulation platforms, such as transcranial magnetic stimulation (TMS) and transcranial direct current stimulation (tcDCS), LIFU has superior temporal and/or spatial resolution. By combining LIFU with existing platforms of stereotaxis, multimodal imaging with MRI, or using the inherent imaging capability of ultrasound, particularly dual-mode ultrasound transducers and arrays, LIFU neuromodulation can target one or many anatomical targets while monitoring therapy.

In one aspect, the present disclosure provides a method including positioning an ultrasound transducer system to direct focused ultrasound energy into a subject's tissue. The method also includes configuring the ultrasound transducer system to direct focused ultrasound energy into target nervous system tissue in the subject's tissue configured to cause heating of the target nervous system tissue to reversibly modulate neural activity. The method also includes delivering focused ultrasound energy to target nervous system tissue based on the configured ultrasound transducer system.

In another aspect, the present disclosure provides a system. The system includes a dual-mode ultrasound transducer configured to deliver focused ultrasound energy. The system also includes a controller operably coupled to the dual-mode ultrasound transducer. The controller is configured to drive the dual-mode ultrasound transducer to deliver focused ultrasound energy to cause heating of target nervous system tissue to reversibly modulate neural activity.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1A illustrates an ultrasound transducer system including an ultrasound transducer array for tFUS thermal neuromodulation.

FIGS. 1B-1D illustrate the delivery of transcranial focused ultrasound using the ultrasound transducer system of FIG. 1A to the ventral posterolateral (VPL) nucleus of the thalamus of a rodent with the mechanical focus highlighted with an oblong oval shape (ultrasound focus) and the larger and more proximate heating profile as a gradient with maximal thermal delivery near the center (thermal focus). Electrical stimulation of the tibial nerve produces signals that travel through the nucleus gracilis, followed by the medial lemniscus to the contralateral ventral posterolateral nucleus of the thalamus, which projects to the somatosensory cortex. Evoked somatosensory-evoked potentials (SSEP) from the VPL with and without ultrasound are shown in FIG. 1E.

FIGS. 2A-2D illustrate image-guided targeting and tFUS beam characterization. FIG. 2A illustrates focus measured in degassed water demonstrating the axial extent of the focus (coronal section). FIG. 2B illustrates B-mode image of the skull at 3 mm behind Bregma with the focal spot placement within the brain (6 mm deep) (asterisk) with midline. FIG. 2C illustrates focus measured in degassed water demonstrating the lateral-elevation profile (axial section). FIG. 2D illustrates a C-scan image of a rodent skull surface from three-dimensional (3D) dual-mode ultrasound array (DMUA) imaging with the active ultrasound wavefront highlighted. The highlighted region illustrates the cross section of the tFUS beam at the skull surface.

FIG. 2E illustrates measured intensity profiles in lateral-elevation plane at 40 mm from the DMUA apex. The upper left profile is the reference measured in the absence of the skull demonstrating the spatial localization in the focal plane. The measured trans-skull profiles in 5 samples demonstrate the reduction in focusing gain (gradient bars), shifting of the focal point (lines) with respect to target (circle), and focus distortion.

FIGS. 3A-3D illustrate a temperature field created by tFUS. FIG. 3A illustrates a spatial profile of the initial heating rate induced by a 1-sec tFUS beam at 50% DC with gradient bar indicating temperature in Celsius and white dot at the target focus. FIG. 3B illustrates montage of tFUS-induced temperature profiles at 0.4 sec intervals (timestamps with respect to tFUS application at 0 sec). The duty cycle of this test shot is 50% rather than the typical 10%. FIG. 3C illustrates steady-state temperature changes plotted across the corresponding duty cycles (displayed in gradients) and amplifier amplitudes (size of the marker) against the SPTA intensity. FIG. 3D illustrates the interpolated temperature surface of the amplitude-duty cycle parameter space plotted over a gradient with reference markers indicating the locations of measured steady-state temperature changes from an image-guided thermocouple.

FIGS. 4A-4D illustrate the effect of LIFU on somatosensory evoked potentials (SSEP) waveforms. FIG. 4A illustrates SSEP waveforms for baseline, contralateral ventral posterolateral nucleus (VPL), and targeted VPL at two intensities with the three main peaks (P1, P2, and P3) and stimulation artifact (S) truncated. FIG. 4B illustrates raw peak-to-peak (P1 to P2) during the course of an experiment (blue bar indicates time when ultrasound is on). FIG. 4C illustrates SSEP waveforms plotted for different ultrasound intensities, measured with I_{SPTA}. FIG. 4D illustrates the ratio of the peak-to-peak SSEP amplitude during ultrasound to baseline plotted against the corresponding SPTA power. Bars indicate means+/−standard deviations of the measured peak-to-peak ratios. A sigmoid function fit to the data with 95% confidence intervals is plotted in dashed gray. (R²=0.98). Electrical stimulation (600 μs) of the tibial nerve was delivered at the zero timepoint denoted in FIGS. 4A and 4C by S where stimulation artifacts are truncated.

FIG. 5 illustrates ultrasound suppression at a constant I_{SPTA}. SSEP peak-to-peak relative to mean baseline and amplitude with 90% confidence bounds are shown.

FIGS. 6A-6B illustrate a timecourse of ultrasound-generated temperature and SSEP change. FIG. 6A illustrates filtered temperature changes in tissue increasing during ultrasound at three different intensities and recovers to baseline over several tens (10's) of seconds. The blue bar indicates time during ultrasound application. FIG. 6B illustrates simultaneous measurement of temperature and SSEP peak-to-peak suppression with tFUS as a function of time. The blue bar indicates time during which tFUS was applied.

FIGS. 7A-7C illustrate laser-mediated thermal neuromodulation. FIG. 7A illustrates temperature change caused by laser heating in thalamus at tip of fiberoptic catheter as a function of time. The red bar indicates time when the laser was applied. FIG. 7B illustrates an average SSEP waveform during baseline, laser neuromodulation, and during the washout or recovery period. Electrical stimulation (600 μs) of the tibial nerve was delivered at the zero timepoint by S in FIG. 7B where stimulation artifacts are truncated. FIG. 7C illustrates a peak-to-peak (P1 to P2) of the SSEP during the laser application. The red bar indicates time when the laser is on.

### DETAILED DESCRIPTION

This disclosure relates to neuromodulation and, in particular, reversible neuroinhibition by focused ultrasound (FUS). LIFU may directly evoke responses and reversibly inhibit function within the brain, or central nervous system. Spatially restricted transcranial ultrasound may provide consistent, inhibitory effects. In this disclosure, the mechanism of reversible suppression in the central nervous system has surprisingly been found to be a thermal mechanism, as others have specifically discounted the role that temperature may play in the etiology of suppression. The present disclosure characterizes the effect and the mechanism of LIFU neuromodulation using a high-precision dual-mode, phased-array ultrasound. For example, transcranial LIFU, or tFUS, was applied to the ventral posterolateral nucleus of the thalamus of a rodent while electrically stimulating the leg (tibial nerve) to induce a somatosensory-evoked potential (SSEP). Thermal changes were also induced through an optical fiber that was image-guided to the same target. LIFU suppressed SSEPs in a spatially and intensity-dependent manner while remaining independent of duty cycle, peak pressure, or modulation frequency. Suppression may be highly correlated and temporally consist with in vivo temperature changes while producing no pathological changes on histology. Furthermore, stereotactically-guided delivery of thermal energy through an optical laser may produce similar thermal effects and suppression. LIFU neuroinhibition may be mediated predominantly by the thermal effect of focused ultrasound. In other words, tFUS predominantly causes neuroinhibition and the primary biophysical mechanism is the thermal effect of focused ultrasound.

When delivering FUS across a skull, the skull may present a significant ultrasound obstacle. The skull is highly reflective, diffractive, and absorptive of ultrasound. It produces a significant barrier to delivery to the central nervous system, and its effects on ultrasound can vary widely across regions of the skull. Phased arrays, modeling, and real-time monitoring may be used overcome the skull as a major barrier to the delivery of focus ultrasound within the brain. Transcranial focused ultrasound (tFUS) may be configured to compensate for these effects in order to minimize distortion, for example, as described in Haritonova, A., Liu, D. & Ebbini, E. S., In Vivo application and localization of transcranial focused ultrasound using dual-mode ultrasound arrays, IEEE Trans. Ultrason. Ferroelectr. Freq. Control 62, 2031-2042 (2015) (hereinafter “Haritonova”), which is incorporated by reference. Phased arrays of ultrasound transducers and modeling may facilitate compensating for anatomical variation in order to focus ultrasound through the skull while distributing energy delivery across the scalp, for example, as described in Kyriakou, A. et al., A review of numerical and experimental compensation techniques for skull-induced phase aberrations in transcranial focused ultrasound, Int. J. Hyperthermia 30, 36-46 (2014) (hereinafter “Kyriakou”), which is incorporated by reference. Real-time monitoring, such as ultrasound thermography, enables imaging of temperature of the tissue heated by ultrasound and can be performed in closed-loop to adjust for distortion, for example, as described in Haritonova and/or Bayat, M., Ballard, J. R. & Ebbini, E. S., Ultrasound thermography in vivo: A new model for calculation of temperature change in the presence of temperature heterogeneity, in 2013 IEEE International Ultrasonics Symposium (IUS) 116-119 (ieeexplore.ieee.org, 2013) (hereinafter “Bayat”), which is incorporated by reference. This technique may successfully produce localized effects in a rodent model through monitoring of tFUS-induced subtherapeutic heating of brain tissues, for example, as described in Darrow D P, Focused Ultrasound for Neuromodulation, Neurotherapeutics 2019; 16:88-99.

At high intensities ultrasound can be used to ablate tissue to create permanent lesions, treat intracranial tumors, or even open the blood-brain barrier. At lower intensities focused ultrasound may reversibly modulate neural activity without damaging tissue. tFUS may even evoke activity or modulate sensory evoked-potentials.

Ultrasound may produce several effects within biological tissue including thermal effects and mechanical effects, such as radiation pressure, shear waves, cavitation, and microcavitation. Some tFUS systems use a single-element ultrasound transducer in a narrowband with a relatively low carrier frequency to minimize losses through the skull. Delivery of ultrasound in this manner may result in tFUS beams with large rostrocaudal extent, allowing interaction with the base of the skull, where the cochlea and inner ear reside, and may produce an auditory-startle effect. In some embodiments, using phased-arrays capable of restricting the ultrasound focus in all three dimensions, or in humans where the skull base remains far from the focus, may result in mostly inhibitory effects on active neural circuits.

The DMUA technology may be used to provide image-guided FUS interventions that facilitate simultaneous delivery of high-resolution therapy while actively monitoring the tissue. The DMUA may provide inherent registration between the imaging and therapeutic coordinate systems, improving both targeting accuracy and safety by minimizing direct exposure to critical structures in the path of the FUS beam. This approach may produce localized application of tFUS, including monitoring of tFUS-induced subtherapeutic heating of brain tissues, for example, as described in Haritonova. In addition, 3D DMUA imaging may provide accurate positioning of the DMUA for repeated application of tFUS, for example, as described in Liu, D., Casper, A., Haritonova, A. & Ebbini, E. S., Adaptive lesion formation using dual mode ultrasound array system, AIP Conf. Proc. 1821, 060003 (2017) (hereinafter “Liu AIP”), which is incorporated by reference. In addition to its basic 3D image guidance capabilities, some DMUA systems employ advanced multi-channel transmit control circuitry that allows use of a large parameter space. For example, multi-channel arbitrary waveform generation may allow the production of tFUS beams with arbitrary temporal modulation in addition to the traditional control of amplitude, duty cycle (DC), and pulse repetition frequency (PRF).

Low-intensity, focused, transcranial ultrasound, or LIFU, may be a useful modality for reversible neuromodulation with high temporal and spatial resolution. Having the ability to inhibit specific neural circuits noninvasively could supplant existing neuromodulation platforms and provide unprecedented access to discover new treatments and understand functional connectivity of a brain in vivo.

LIFU can be used to suppress a primary sensory pathway by applying it to the thalamus in a rodent. The slow temporal dynamics of this inhibition, which may build over 10's of seconds, may indicate that the main effect of the ultrasound at these intensities may not be a direct modulation of the excitability of the neurons. Rather, the inhibition may be sigmoidally related to the applied power or time average ultrasound intensity. Moving the ultrasound focus by a few millimeters off-target may result in no significant suppression, demonstrating a spatially-restricted effect of the ultrasound delivered using a DMUA.

Different frequencies and pulse widths may be used to find parameters of sinusoidal ultrasound (US) that is optimized for neuroinhibition. The DMUA system may or may not use refocusing to provide neuroinhibition to target tissue. For example, when the VPL of the thalamus is used as a target for FUS, even though a DMUA system may refocus, targeting the size and location of the VPL may need very little refocusing. In general, DMUA systems may provide tremendous flexibility for applying ultrasound for thermal nueromodulation.

In some embodiments, the focus of ultrasound energy from the ultrasound system may be limited in size. The focus of the ultrasound energy may be focused to avoid certain parts of the brain. For example, the focus may be sized and directed to avoid an acoustic startle response, which may be caused by large ultrasound interacting with the skull base, where the mechanical transduction system of the ear is rigidly housed. In some embodiments, a DMUA system may be used to prevent significant thermal or mechanical energy from being delivered distal to a target, for example, by selecting the appropriate operating frequency and geometry of the transducer.

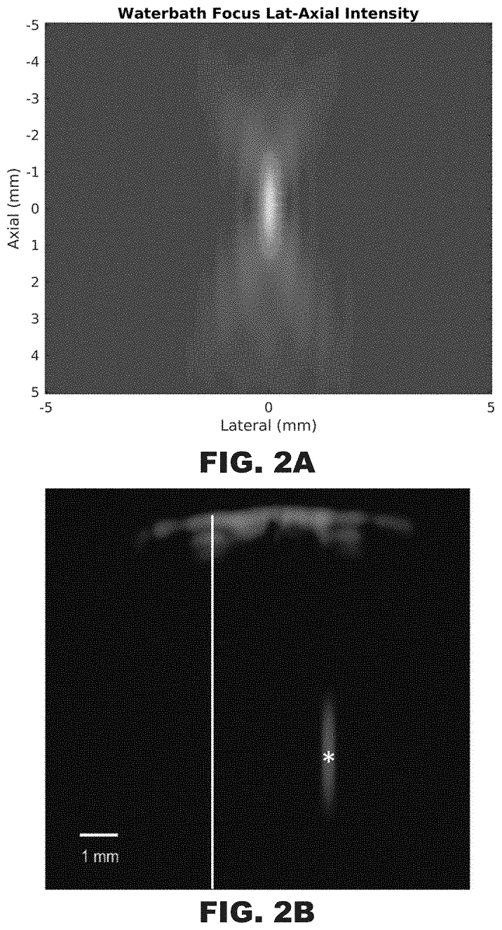

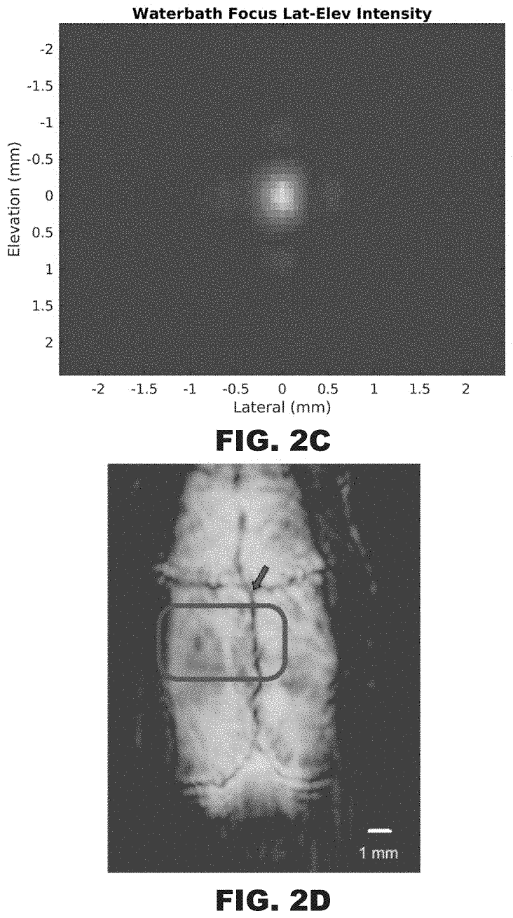

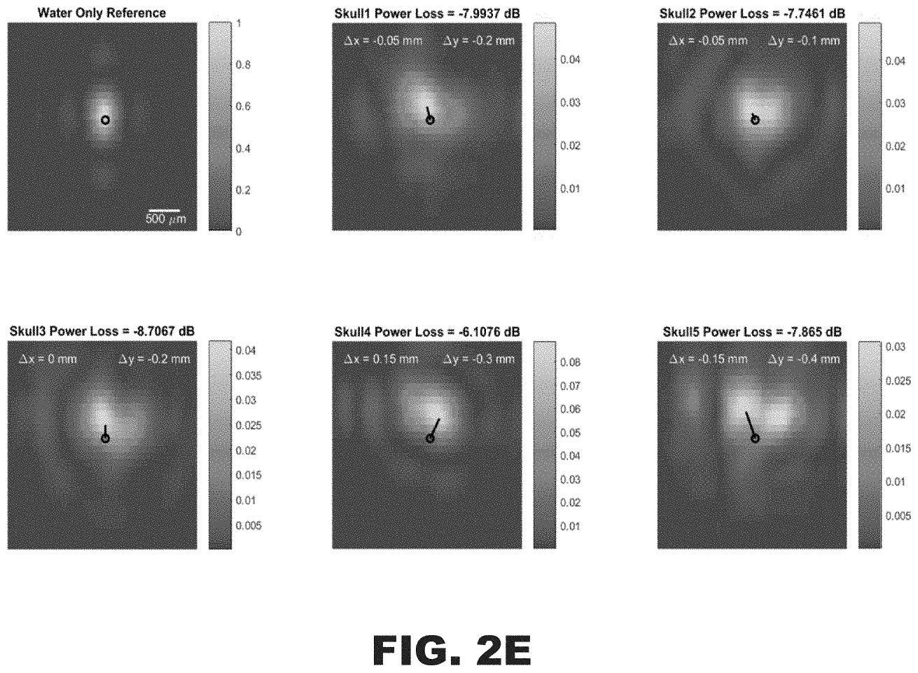

Ultrasound delivered to a skull may be configured to be spread over a large surface as shown, for example, in FIG. 2. The operating frequency and geometry of the transducer may be selected to optimally spread the ultrasound over such a large surface.

Some ultrasound delivered may cause prefocal heating. As a result, brain tissues proximal to the target may receive some thermal effects. The extent of the affected brain tissue volume may depend on the actual exposure duration and the duty cycle. For example, when targeting the thalamus, the affected volume may have a pyramid shape extending from just below the cortex (e.g., 3 to 4 mm wide) with an apex at the target. Thermal suppression of the SSEP may be a result of white matter and/or grey matter suppression from the thalamus to the cortex. Refinement of the affected volume may be achieved by refocusing the tFUS beam to compensate for distortion through the skull and the design of site-specific DMUA applicators.

Inhibition with ultrasound may depend primarily on intensity rather than the pressure-wave amplitude or duty cycle. In other words, reproducible suppression of SSEP is dependent on the total energy delivered in a dose of ultrasound intensity. The dose may be modulated using amplitude and duty cycle.

In some embodiments, saturated SSEP suppression may be achieved with temperature changes of approximately 2 degrees, which has been validated by measurement with ultrasound thermography and fine wire thermocoupling, where resolution is around 0.1 degrees C. The temperature produced at the focus may be highly correlated with the intensity, and the intensity may be proportional to the temperature. Temperature may cause or correlate to inhibition. Thermal changes were applied focally with ultrasound and with a laser with similar results. Thermal from non-thermal effects of ultrasound may be interdependent, as supported by the cross-modal validation of the thermal effect.

Thermal neuromodulation in a restricted focus through a noninvasive technique may be applied to treatment of diseases of the central nervous system. Noninvasive LIFU may not be restricted to a single focus and may be used in a closed-loop application. Noninvasively heating a spatially-restricted volume of neural tissue without damage may provide a method of controlling networks through multiple foci and investigating the basis of disease and neural function.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar.

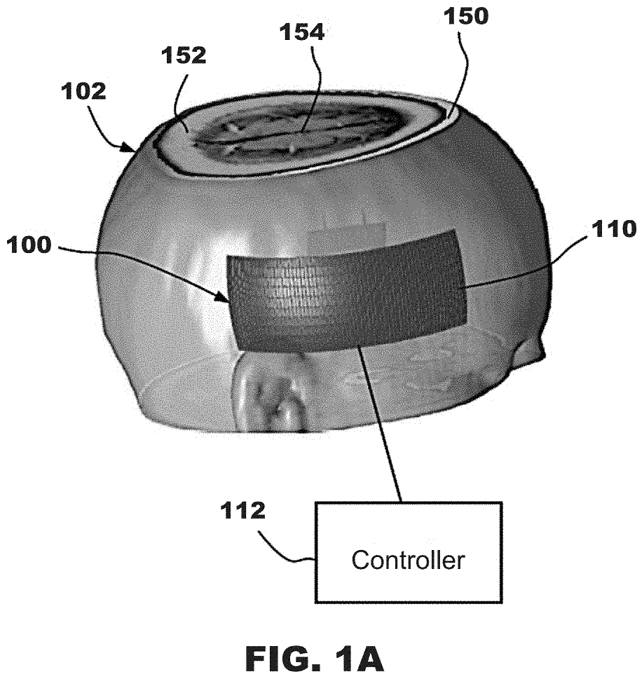

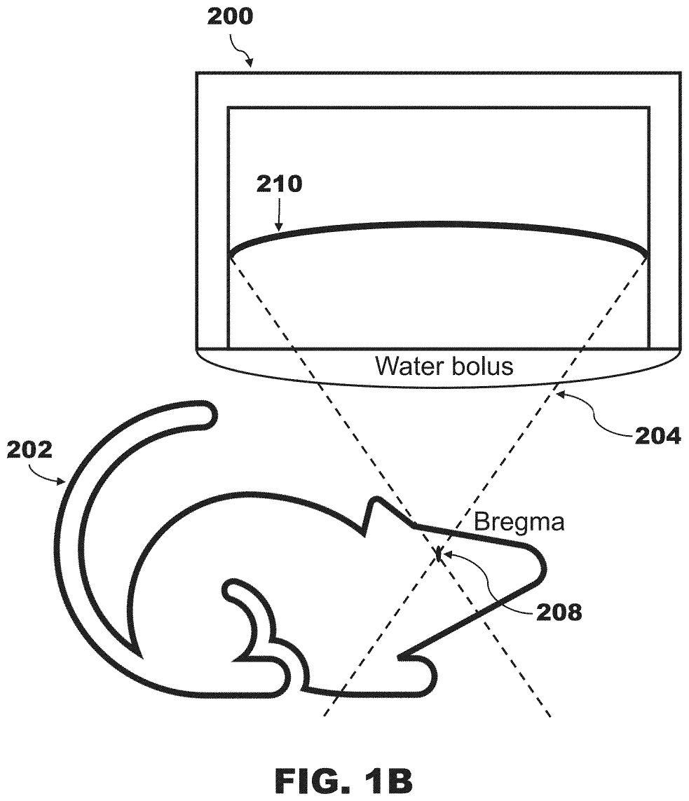

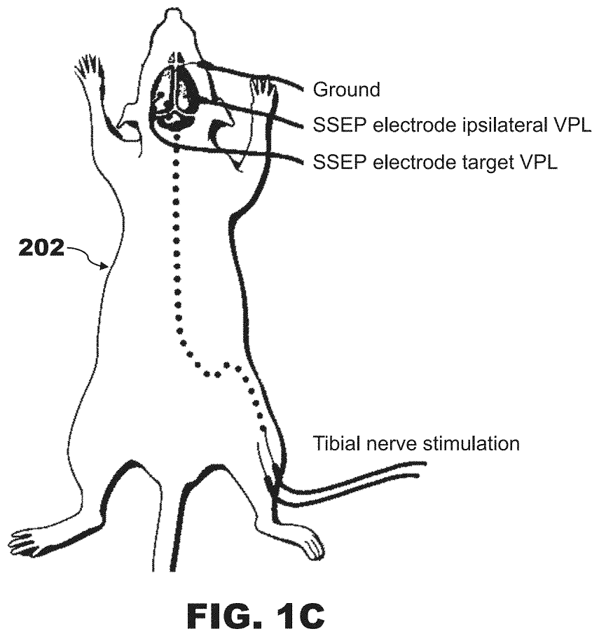

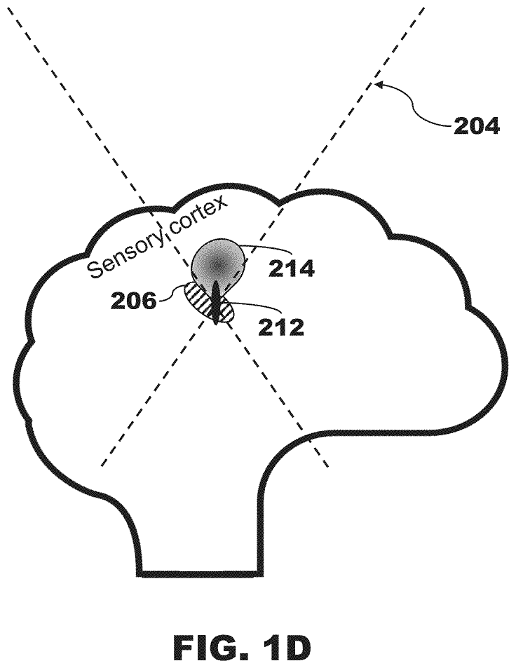

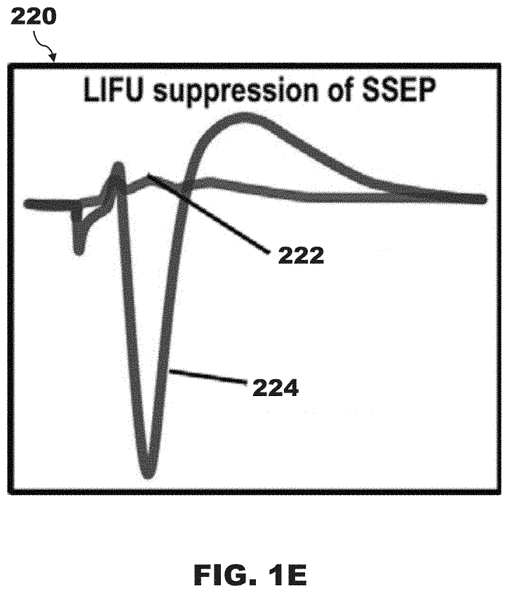

FIGS. 1A-1D show an ultrasound transducer system 100 used with a human head 102 and with a rodent 202. FIG. 1A is a perspective view of an ultrasound transducer system 100 sized and configured for use with the human head 102. FIG. 1B is a perspective view of an ultrasound transducer system 200 sized and configured for use with the head of the rodent 202, and FIG. 1C is an overhead view of a tFUS beam 204 generated by the ultrasound transducer system 100 applied to the rodent 202. FIG. 1D shows an elevational cross-section showing the tFUS beam 204 and the brain of the rodent 202. The tFUS beam 204 may be used in a similar manner to be applied to the human head 102 of FIG. 1A.

Ultrasound transducer system 100 may include transducer array 110, or transducer portion or patch, which is shown attached to subject 102 (e.g., a patient). As illustrated, transducer array 110 is coupled adjacent to surface layer 150 (e.g., skin/scalp) of the head of subject 102. The ultrasound transducer system 100 may include controller 112 (e.g., a control circuit or circuitry) operably coupled to transducer array 110. A cross-section of head 102 is shown for illustrative purposes, in particular, to show the subject's ultrasound obstacle 152 (e.g., a skull, which may cause ultrasound distortion) and subject tissue 154 (e.g., brain tissue and/or nervous system tissue). Description of the ultrasound transducer system 100 and the transducer array 110, is generally applicable to the ultrasound transducer system 200 and the transducer array 210.

System 100 may be configured to provide delivery, monitoring, and control of localized tFUS and may use a light-weight, conformable transducer array. System 100 may be optimized for targeting specific circuit(s) within the brain utilizing low-power drivers and processors, which may be included in the controller 112, for closed-loop control of focused ultrasound energy deliver for neuromodulation. Transducer array 110 may be described as a DMUA comprising one or more dual-mode ultrasound transducers.

Ultrasound transducer system 100 may be described as a tFUS applicator and may be customized to each subject to optimize ultrasound energy deposition in a small target volume (e.g., at target points in a target volume).

In some embodiments, controller 112 includes front-end circuitry that may be used for full-duplex DMUA operation, which may improve localization of heating while tFUS thermal neuromodulation is active. The front-end circuitry may also be used for transmitting waveforms with very large time-bandwidth product (e.g., large bandwidth and/or long duration), which may also have appropriate, or desirable, correlation properties (e.g., orthogonal or almost orthogonal waveforms) for improved spatial localization in the axial direction (e.g., a direction orthogonal to a transducer major surface or substantially parallel to the direction of propagation of the transmit ultrasound wavefront).

In general, the transducer array 110 of ultrasound transducer system 100 may be positioned in any suitable location and orientation to direct focused ultrasound energy to target nervous system tissue within the tissue of subject 102. As illustrated, transducer array 110 of ultrasound transducer system 100 is positioned outside of the skull of subject 102 and outside of the nervous system tissue of subject 102. In some embodiments (not shown), transducer array 110 of ultrasound transducer system 100 may be at least partially or entirely positioned within a bore hole in the skull while remaining outside of the tissue of subject 102. In some embodiments (not shown), transducer array 110 of ultrasound transducer system 100 may be at least partially or entirely positioned within the patient's tissue, such as the patient's brain tissue.

Low-intensity tFUS neuromodulation, or non-transcranial LIFU when the transducer array 110 is at least partially positioned within the skull or tissue of subject 102, may be used on target nervous system tissue of subject 102.

FIGS. 1B-1D show the ventral posterolateral (VPL) nucleus 206 of the thalamus of a rodent with the mechanical focus highlighted with an oblong oval shape (ultrasound focus 212) and the larger and more proximate heating profile as a gradient with maximal thermal delivery near the center (thermal focus 214). The ultrasound focus 212 and the thermal focus 214 are produced by the transducer array 210 generating the tFUS beam 204, which interacts with the tissue of the rodent 202. Electrical stimulation of the tibial nerve produces signals that travel through the nucleus gracilis, followed by the medial lemniscus to the contralateral ventral posterolateral nucleus of the thalamus, which projects to the somatosensory cortex. FIG. 1E shows a plot 220 of evoked somatosensory-evoked potentials (SSEP) from the VPL with tFUS ON 222 and with tFUS OFF 224 ultrasound.

Various conditions may be treated using thermal neuromodulation. In general, any functional disease of the nervous system may be treated using FUS, for example, in the brain or other parts of the nervous system (e.g., peripheral nerves). Non-limiting examples of conditions include epilepsy, pain, movement disorders (e.g., Parkinson's, essential tremor, etc.), psychiatric diseases (e.g., depression, anxiety, obsessive-compulsive disorder, etc.), and mapping of brain tumors (e.g., to determine areas for safe surgery). In particular, non-invasive treatment of these conditions may be facilitated by tFUS by directing ultrasound energy to regions or parts of the brain associated with these conditions.

In some embodiments, tFUS may be delivered to the thalamus using a mechanical focus and a larger focus (extending beyond the mechanical focus where the temperature of the tissue is increased) on maximal delivery of thermal energy. For example, electrical stimulation of the tibial nerve may produce an SSEP that travels through the nucleus gracilis, followed by the medial lemniscus to the contralateral VPL nucleus of the thalamus, which projects to the somatosensory cortex. Evoked SSEPs may be inhibited due to the effects of delivering LIFU energy.

In some embodiments, transducer array 110 of system 100 may include a dual-mode ultrasound transducer configured to deliver FUS to target nervous system tissue. Controller 112 may be configured to drive the dual-mode ultrasound transducer, or multiple dual-mode ultrasound transducers of transducer array 110, to deliver FUS to cause heating of target nervous system tissue to reversibly modulate neural activity.

Transducer array 110 may be a dual-mode ultrasound array. For example, transducer array 110 may include a dual-mode ultrasound transducer and at least one other dual-mode ultrasound transducer. Both may be used cooperatively to provide the focused ultrasound energy.

Each transducer may be positioned in a different location relative to subject 102. The transducers may also be implanted or attached at the same or different levels of invasiveness. For example, one or more transducers of transducer array 110 may be positioned outside of the skull of subject 102 and one or more other transducers of transducer array 110 may be positioned within the skull or tissue of subject 102.

Ultrasound transducer system 100 may be configured to heat the target nervous system tissue an appropriate amount to provide neuromodulation, such as inhibition of neural activity. In some embodiments, the focused ultrasound energy may be configured to heat the target nervous system tissue greater than or equal to about 0.1, 0.2, 0.3, 0.5, 1, 2, or 3 degrees Celsius (° C.) at any location within the ultrasound field of view. In some embodiments, focused ultrasound energy may be configured to heat the target nervous system tissue less than or equal to about 3, 2, 1, 0.5, 0.3, 0.2, or 0.1° C. at any location within the ultrasound field of view.

Successful neuromodulation may be based on an appropriate intensity of FUS reaching the target nervous system tissue. FUS intensity may be highly correlated to heating of tissue. One important measure of FUS intensity related to heating is spatial-peak, temporal-average intensity (I_{SPTA}). In general, I_{SPTA} of the delivered FUS energy may be determined such that the target nervous system tissue is undamaged, for example, on histology, after delivering the FUS. In some embodiments, the I_{SPTA} of the delivered FUS at the target nervous system tissue is greater than or equal to about 0.1, 0.2, 0.3, 0.5, 1, 2, 3, 4, 5, 10, 20, 30, 40, 50, 60, 70, or 80 W/cm². In some embodiments, the I_{SPTA}of the delivered FUS at the target nervous system tissue is less than or equal to about 80, 70, 60, 50, 40, 30, 20, 10, 5, 4, 3, 2, 1, 0.5, 0.3, 0.2, or 0.1 W/cm². In some embodiments, for delivering ultrasound energy in some human patients, the I_{SPTA} may be less than or equal to about 30 or 40 W/cm². Measures of FUS intensity other than I_{SPTA} may be less correlated to heating effects desirable in thermal neuromodulation.

In some embodiments, power (P_{SPTA}) may be derived from I_{SPTA}, for example, relative to a baseline. In some embodiments, P_{SPTA} may be greater than or equal to about 0.5, 1, or 1.5 dB W/cm² and/or less than or equal to about 0.5, 1, or 1.5 dB W/cm².

Intensity of the FUS may depend on various parameters, such as duration of energy delivery (e.g., shot duration), duty cycle, and carrier frequency. FUS energy may be delivered using ultrasound pulses having a duty cycle.

In general, the duration of energy delivery may be any suitable duration for causing the appropriate amount of heating for thermal neuromodulation. The duration used to heat tissue with FUS may be longer than a duration that would be used, for example, to cause cavitation. In some cases, heating with FUS uses durations on the order of several seconds, or even 10's seconds. In some embodiments, FUS energy is delivered for greater than or equal to about 1, 2, 3, 5, 10, 20, or 30 seconds. In some embodiments, FUS energy is delivered for less than or equal to about 30, 20, 10, 5, 3, 2, or 1 seconds. In some embodiments, FUS energy may be delivered as long as thermal neuromodulation is desired (e.g., all day for ambulatory therapy).

Duty cycle may be related to duration to achieve the desired FUS intensity. The duty cycle for heating with FUS may be higher compared to other applications, for example, using FUS to cause cavitation. In some embodiments, the FUS has a duty cycle greater than or equal to about 1, 2, 3, 4, 5, 6, 7, 8, 9, or 10%. In some embodiments, the FUS has a duty cycle less than or equal to about 10, 9, 8, 7, 6, 5, 4, 3, 2, or 1%.

In general, carrier frequency may be any suitable carrier frequency for achieving a desired focus size that includes the target nervous system tissue while bypassing various obstacles, such as the skull or other tissue. For example, tFUS may use higher frequencies compared to non-transcranial FUS, so the position of the transducers may be selected to cooperate with the selected carrier frequency. In particular, when using non-transcranial FUS, the carrier frequency may be arbitrary, or within a wide range of frequencies, such as any frequency up to 3.2 megahertz (MHz). In some embodiments, the FUS has a carrier frequency greater than or equal to about 1, 1.5, 2, 2.5, or 3 MHz. In some embodiments, the FUS has a carrier frequency less than or equal to about 3, 2.5, 2, 1.5, or 1 MHz. Further, in some embodiments, multiple carrier frequencies may be used to deliver FUS energy. Using multiple carrier frequencies may facilitate delivering FUS to the target nervous system tissue while bypassing obstacles, such as the skull.

Using a DMUA for tFUS or non-transcranial FUS may allow the focal spot size of delivered FUS to be constrained to very small volumes. For example, the focal spot size may be constrained on the single-digit millimeter level in one, two, or three dimensions. On the other hand, the focal spot size may also be enlarged to several millimeters, or even centimeters, for example, to target the entire temporal lobe of subject 102. In some embodiments, the focal spot size may be greater than or equal to about 1, 1.25, 1.5, 2, 3, 4, 5, 6, 7, 8, 9, 10, 20, or 30 millimeters (mm) in one, two, or three dimensions. In some embodiments, the focal spot size may be less than or equal about 30, 20, 10, 9, 8, 7, 6, 5, 4, 3, 2, 1.5, 1.25, or 1 mm.

In some embodiments, the DMUA transducer may be concave to reduce the size of the tFUS focus to a fraction of a millimeter in the lateral direction and approximately 2 mm in the dorsoventral (axial) direction. Distortion from the skull may extend the focal spot size in the lateral-elevation dimensions. Even with these distortions, the focal spot may a fraction of, or less than, that used in other systems, which may be the size of 3.5 mm lateral and 6.2 mm along the beam axis. In general, a small tFUS focus may facilitate precise placement of the tFUS beam with reference to the target, as well as monitoring the effects of tFUS, e.g., through real-time thermography.

Using a DMUA, multiple focal spots may also be used. For example, the ultrasound transducer system 100 may be configured to target multiple focal spots within a single target nervous system tissue or may target multiple target nervous system tissues. In some embodiments, two, three, four, or more focal spots may be used concurrently.

The focal spot may be static or dynamic. In some embodiments, the focal spot may be permanently located in the subject's tissue. In some embodiments, the focal spot may move over time. For example, the focal spot may be rasterized in one, two, or three dimensions to cover a target tissue system volume that is larger than the focal spot size.

One or more of the components, such as controllers or arrays, described may include a processor, such as a central processing unit (CPU), computer, logic array, or other device capable of directing data coming into or out of ultrasound transducer system. The controller may include one or more computing devices having memory, processing, and communication hardware. The controller may include circuitry used to couple various components of the controller together or with other components operably coupled to the controller. The functions of the controller may be performed by hardware and/or as computer instructions on a non-transient computer readable storage medium.

The processor of the controller may include any one or more of a microprocessor, a microcontroller, a digital signal processor (DSP), an application specific integrated circuit (ASIC), a field-programmable gate array (FPGA), and/or equivalent discrete or integrated logic circuitry. In some examples, the processor may include multiple components, such as any combination of one or more microprocessors, one or more controllers, one or more DSPs, one or more ASICs, and/or one or more FPGAs, as well as other discrete or integrated logic circuitry. The functions attributed to the controller or processor may be embodied as software, firmware, hardware, or any combination thereof. While described as a processor-based system, an alternative controller could utilize other components such as relays and timers to achieve the desired results, either alone or in combination with a microprocessor-based system.

In one or more embodiments, the exemplary systems, methods, and interfaces may be implemented using one or more computer programs using a computing apparatus, which may include one or more processors and/or memory. Program code and/or logic described may be applied to input data/information to perform functionality described and generate desired output data/information. The output data/information may be applied as an input to one or more other devices and/or methods as described or as would be applied in a known fashion. In view of the above, it will be readily apparent that the controller functionality as described may be implemented in any manner known to one skilled in the art.

While the present disclosure is not so limited, an appreciation of various aspects of the disclosure will be gained through a discussion of the specific examples and illustrative embodiments provided below, which provide alloys with superior mechanical and corrosion properties. Various modifications of the examples and illustrative embodiments, as well as additional embodiments of the disclosure, will become apparent.

### EXAMPLE

First, the DMUA focus and the effect of the rodent skull on the focus were characterized. Then, the effects of low-intensity, focused, transcranial ultrasound on somatosensory-evoked potentials generated by electrical stimulation of the tibial nerve in a rat were tested. LIFU was varied in amplitude, modulation frequency, and duty cycle and the effect of each parameter on an SSEP was measured. Lastly, after finding evidence for the role of thermal energy as a correlate of inhibition, similar suppression through the effect of focal heating with light applied through an optical fiber was tested, providing cross-modal evidence implicating temperature as the predominant mechanism of LIFU neuroinhibition.

A carrier frequency of 3.2 MHz in the DMUA system was used, allowing a precise level of localization over single transducer systems working around 690 kHz, such as the system described in Min, B.-K. et al. Focused ultrasound-mediated suppression of chemically-induced acute epileptic EEG activity. BMC Neurosci. 12, 23 (2011) (hereinafter “Min”). In addition, the DMUA transducer was highly concave, which reduced the size of the tFUS focus to a fraction of a millimeter in the lateral/axial direction and approximately 2 mm in the rostrocaudal direction (FIGS. 2A & 2C). The distortion by the skull extended the focal spot size in the lateral-elevation dimensions (FIG. 2B). Even with these distortions, the extent of the focal spot was a fraction of the focal spot produced by the transducer used by Min, which was 3.5 mm in the lateral dimension and 6.2 mm along the beam axis, for example.

Volumized ultrasound scans allowed for reliable targeting of stereotactic targets with high precision. The inherent registration between the imaging and therapy coordinate systems in the DMUA system was taken advantage of when placing the tFUS beam with reference to the target and when monitoring its effects, e.g. through real-time thermography. The DMUA system offered high temporal and spatial resolutions in monitoring and delivery of tFUS beams in both thermal and mechanical modes, which can be used for closed-loop control of exposure, as demonstrated in Casper, A. J., Liu, D., Ballard, J. R. & Ebbini, E. S., Real-time implementation of a dual-mode ultrasound array system: In vivo results, IEEE Transactions on Biomedical Engineering 60, 2751-2759 (2013) (hereinafter “Casper”), which is incorporated by reference.

Some results of the experiment may be summarized as follows: The FUS focus size was confirmed using ex-vivo skulls. Reproducible suppression of the SSEP proportional to US intensity was observed. Combinations of amplitude and duty cycle were tested, but suppression was mainly correlated with total energy delivered and no significant difference in the effect when duty cycle was changed but the intensity was held constant. The effect of FUS was generally not instantaneous, taking 10's of seconds for onset of maximal effect, and persisting for 10's of seconds after cessation of the therapy. The time course was similar mainly to the thermal time constant caused by ultrasound heating. Intensity was found to be linearly correlated with the steady-state temperature. Recovery of the evoked potential to baseline after washout and unremarkable histological changes may indicate that FUS intensity needed to suppress the neural activity may not cause irreversible damage.

Ultrasound System

A 64-element DMUA transducer was used for transcranial imaging and delivery of tFUS neuromodulation as described in Haritonova. The array was driven by a 32-channel linear amplifier with programmable independent waveforms in both imaging and therapy modes. The arbitrary driving waveforms were synthesized based on the desired target (focus) point and modulation scheme using previously described focusing algorithms as described in Ebbini, E. S., Yao, H. & Shrestha, A., Dual-mode ultrasound phased arrays for image-guided surgery, Ultrason. Imaging 28, 65-82 (2006) (hereinafter “Ebbini USI”), which is incorporated by reference. A custom designed MATLAB (R2016a, The MathWorks, Inc., Natick, Mass.) interface was used to download the waveforms to a custom FPGA front end as described in Casper.

Ultrasound Imaging

Two DMUA imaging modes, described previously, were used. Briefly, synthetic aperture (SA) imaging mode was used for guidance and localization of tFUS. as described in Liu, D., Schaible, K., Low, W. & Ebbini, E. S., Three-dimensional image guidance for transcranial focused ultrasound therapy, 2017 IEEE 14th International Symposium on Biomedical Imaging (ISBI 2017) 916-919 (ieeexplore.ieee.org, 2017) (hereinafter “Liu IEEE”), which is incorporated by reference. Single transmit focus (STF) imaging was used for monitoring of tFUS application and to characterize its thermal and mechanical bioeffects as described in Wan Y, Ebbini E S., Imaging with concave large-aperture therapeutic ultrasound arrays using conventional synthetic-aperture beamforming, IEEE Trans Ultrason Ferroelectr Freq Control 2008; 55:1705-18 (hereinafter “Wan”). STF is a high speed imaging mode that allowed for monitoring the tissue response to tFUS at hundreds of frames per second.

Ultrasound Characterization

Power and intensities were measured in a degassed water bath with and without ex-vivo skulls using an Onda hydrophone (Onda Corporation, Sunnyvale, Calif.). Field scans were performed in planes parallel to the surface of the DMUA in the focal plane of the intended focus, i.e. to measure a cross section of the focal spot. This was done using a fine grid with spacing smaller than λ/3, where λ is the wavelength of the tFUS beam. The extent of the scan was large enough to capture the 2D extent of the focal spot and the significant sidelobes in the lateral and elevation directions. Using calibration tables provided by the vendor, this allowed the use of the hydrophone measurements to compute the acoustic pressure, intensity and integrated power.

Ultrasound Thermography

Briefly, 1-sec test tFUS shots were delivered to the target at intensity levels similar to those used in the neuromodulation experiments described in this paper. Real-time DMUA imaging data was collected 1 sec before, during, and 8 sec after tFUS at 200 frames per second. The signal processing algorithm as described in Ebbini, E. S., Simon, C. & Liu, D. Real-Time Ultrasound Thermography and Thermometry [Life Sciences]. IEEE Signal Process. Mag. 35, 166-174 (2018) (hereinafter “Ebbini IEEE”), which is incorporated herein by reference in its entirety, was used to evaluate the temperature change profiles resulting from the application of the 1-sec test shots.

Temperature Characterization

A type-T (copper-constantan) 200 μm hypodermic needle thermocouple (Type-0, Omega Engineering, Norwalk, Conn.) was used for localized measurement of temperature at the target location. The temperature values were measured at a rate of 100 Hz using Agilent 34970A data acquisition unit (Agilent Technologies, Santa Clara, Calif.).

Stereotaxis and 3D Rendering

A Daedal xyz-stage computer-controlled stepper-motor system (Parker-Hannifan, Cleveland, Ohio) with precise three-dimensional control was used. The xyz-stage was used to obtain sequential coronal SA DMUA images. A custom designed FPGA controller was used to synchronize the motion position with the SA frame count to allow for 3D rendering of the volume scan data. Transverse planes corresponding to C-mode ultrasound scan were extracted for identification of the suture lines, which were used as reference for precise placement of the tFUS beam using a method of stereotaxis as described in Liu IEEE. Three-dimensional DMUA imaging was used to position the focus at precise stereotactic coordinates with respect to the skull sutures, which were easily identifiable with imaging (see FIG. 2D). The ultrasound focus to the ventral posterior lateral (VPL) nucleus of the thalamus (3 mm posterior to Bregma, 3 mm lateral, 6 mm deep to cortex) was targeted as described in Paxinos, G. & Watson, C. The rat brain atlas in stereotaxic coordinates. San Diego: Academic (1998) (hereinafter “Paxinos”), which is incorporated by reference.

SSEP Experimental Procedure

In accordance with a UMN-approved IACUC protocol, Sprague-Dawley (obtained from Charles River Laboratories) rats (n=15) between 250 g and 350 g were anesthetized with a ketamine (40-90 mg/kg) and xylazine (5-10 mg/kg) delivered IP until complete suppression of motor response to deep toe pinch. Maintenance injections of ketamine (20 mg/kg) were delivered if a toe pinch response was measured. No effort was made to lighten anesthesia. The head was rigidly fixed with ear bars, and 1 cc of 1% lidocaine with 1:100000 epinephrine was used for local anesthetic. The scalp was incised and periosteum cleared. One mm burr holes were placed bilaterally at 2 mm posterior and 2 mm lateral to Bregma. Epidural potentials were measured via the burr holes using shielded stainless steel wire with differential source from a midline subcutaneous frontal electrode and separately grounded subcutaneously. Signals were amplified 100× and bandpass filtered between 0.1 Hz and 3 kHz using Grass P511 amplifiers. Signals were then digitized at 10 kHz using a National Instruments (Austin, Tex.) 16-bit digitizer. Data was recorded using open source Real-Time eXperimental Interface software as described in as described in Patel, Y. A. et al., Hard real-time closed-loop electrophysiology with the Real-Time eXperiment Interface (RTXI), PLoS Comput. Biol. 13, e1005430 (2017) (hereinafter “Patel”). The tibial nerve was stimulated after insertion of subcutaneous needles, using a voltage-controlled GRASS (S88, Grass Astro-Med, W. Warwick, R.I.) stimulator at 4.3 Hz and pulse width of 600 μsec. A voltage between 0.5 and 3 V was tailored to generate the minimal suprathreshold stimulation.

Experimental epochs consisted of 120 s of baseline SSEP followed by 150 s of SSEP during ultrasound stimulation, which was followed by a 60 s washout period. Baseline SSEP measurements were taken to calculate 10 sequential periods of 50 SSEP windows. Ultrasound was delivered for 30 seconds followed by 10 sequential periods of 50 SSEP windows. Average SSEP waveforms were calculated during each period. Peak-to-peak measurements were computed for each period. To provide a conservative estimate of ultrasound's effect on the SSEP, the ratio of every combination of baseline and therapy windows were calculated and used as the distribution to determine the effect size across ultrasound parameters.

Laser Heating

A 50 mW laser of 650 nm (Visual Fault Locator, J-Deal TL532) was used to deliver light through a 100 m8icrometer (μm) fiberoptic cable. Incident light calibration measured 35 mW at the distal tip.

Statistical Analysis

The Kolmogorov-Smirnov Test was used to compare groups of data. Alpha was selected to be 0.05 with Bonferroni correction for multiple comparisons.

Ultrasound Characterization

To deliver ultrasound, a 64-element DMUA prototype, as described in Haritonova, was used to generate modulated tFUS patterns in the thalamus with the DMUA prototype shown in FIGS. 1B-1D. The transmitted FUS field patterns were first measured in degassed water. Measurements were made using a 3.2 MHz carrier frequency, 50 kHz modulation frequency, and pulse-repetition frequency of 500 Hz in saline. Reference focal plane patterns were measured on a grid with 0.05 (medial-lateral)×0.1 (elevation) mm² spacing in five ex-vivo calvaria as shown in FIGS. 2A and 2C.

The DMUA was positioned with respect to the intersection of the bregma and medial suture lines (arrow in FIG. 2D). The intersection of the bregma and medial suture lines (arrow) served as a marker for placing the DMUA for targeting the stereotactic coordinates.

In each experiment, a 3D image of the skull surface was obtained by mechanically scanning the DMUA while collecting 2D pulse-echo data in synthetic aperture (SA) imaging mode as described in Liu, D., Schaible, K., Low, W. & Ebbini, E. S. Three-dimensional image guidance for transcranial focused ultrasound therapy. in 2017 IEEE 14th International Symposium on Biomedical Imaging (ISBI 2017) 916-919 (ieeexplore.ieee.org, 2017) (hereinafter “Liu IEEE”), which is incorporated herein in its entirety. To characterize beam distortion due to skull interference, the field patterns were measured transcranially on the same focal plane grid using 5 skull samples.

These measurements revealed significant loss in transmitted power due to absorption by the skull. FIG. 2B shows the focus below the skull in a coronal section. On average, 15% of the FUS beam peak power (8 dB loss with variance of 1.3 dB) was transmitted through the skull to the target location. The width of the beam at 3 dB reduction from the peak power was found to be deformed (from a resolution of 0.5 mm lateral diameter by 1 mm in the orthogonal direction in water) to 1 mm in the lateral direction by 2.5 mm in the orthogonal direction at the stereotactic coordinates of the VPL. In addition, defocusing effects were evident in some cases, including split focusing.

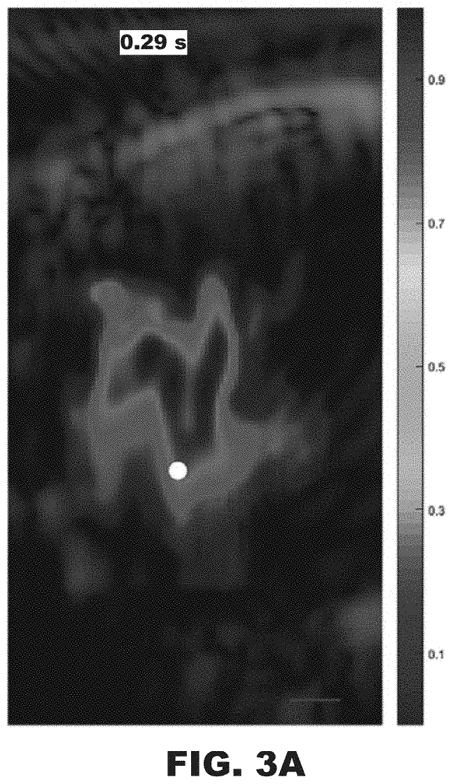

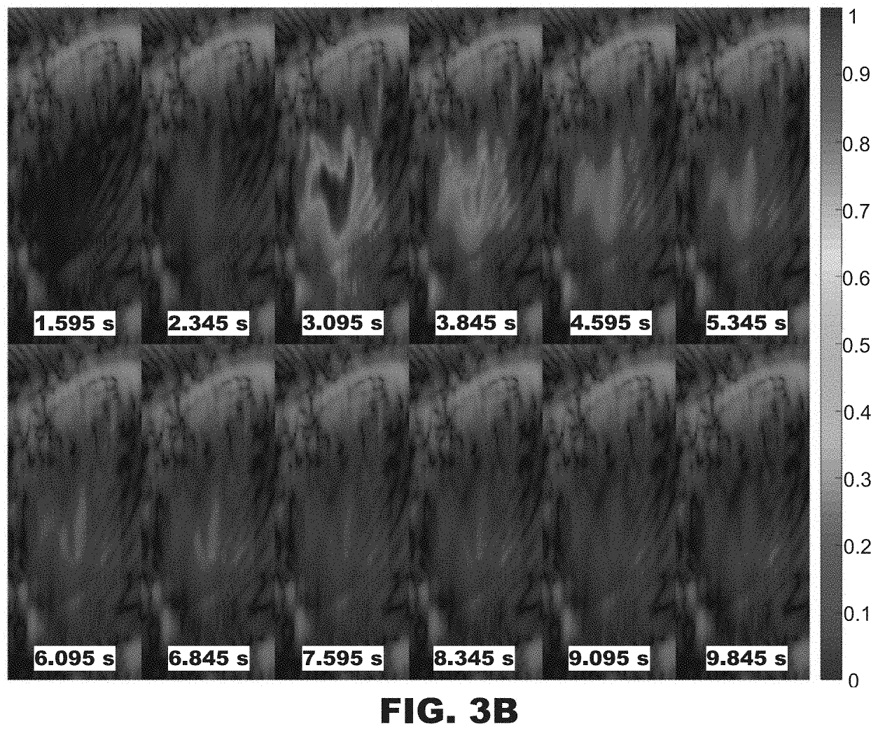

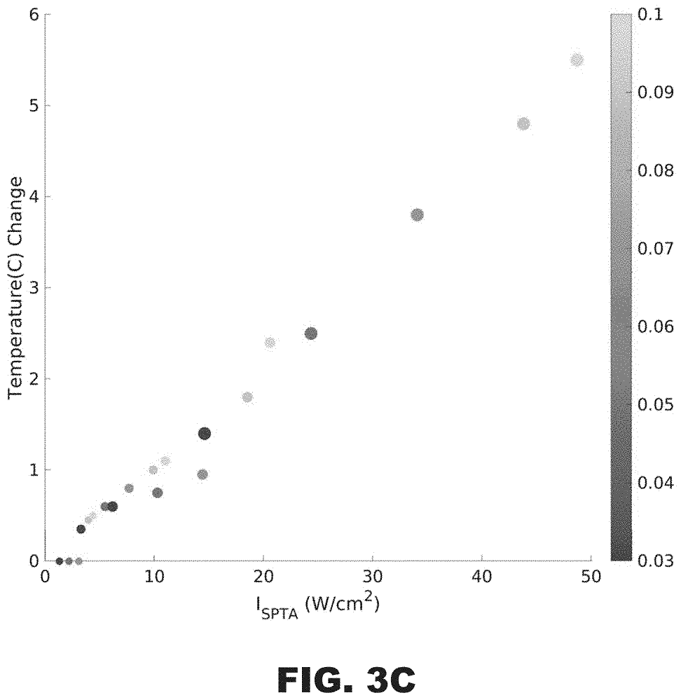

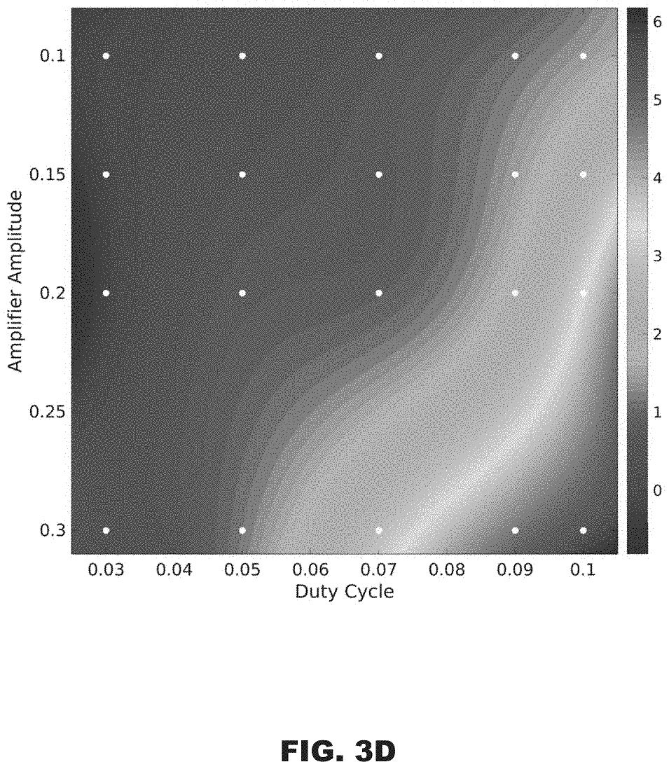

Real-time ultrasound thermography was used to measure the temperature and spatial-temporal evolution of the temperature field due to tFUS in vivo similar to described in Ebbini, E. S., Simon, C. & Liu, D. Real-Time Ultrasound Thermography and Thermometry [Life Sciences]. IEEE Signal Process. Mag. 35, 166-174 (2018) (hereinafter “Ebbini IEEE”), which is incorporated herein by reference in its entirety. FIG. 3A shows the heating rate of the tFUS beam, which is indicative of the spatial intensity distribution in coronal section, and FIG. 3B illustrates the time-evolution of the temperature field with respect to tFUS-on time at 1-sec intervals during a characterization test. The temperature profiles demonstrate the spatial localization of tFUS-induced heating consistent with intensity profiles shown in FIGS. 2A-2D and the distortion patterns shown in FIG. 2E. Specifically, the maximum tFUS-induced heating occurred approximately 1 mm prefocally (or 1-1.5 mm prefocally, due to the lensing effect of the skull) with an extent of approximately 3 mm in the beam direction. The heated region has a classic “tadpole” shape with the extent of the heated region in the orthogonal direction of approximately 2 mm (or 2.4 mm) at the widest point.

The thermal effect in vivo was systematically evaluated across a broad range of ultrasound amplitudes and duty cycles using a fine-needle thermocouple temperature probe. A temperature response surface was interpolated, as shown in FIG. 3D. As expected, the temperature increased with the square of the amplitude of the ultrasound and linearly with the duty cycle. The change in temperature was highly correlated (Spearman R 0.96) with the spatial peak temporal average intensity, I_{SPTA}, as can be seen supplemental FIG. 3C.

tFUS Modulation of SSEP Amplitude

To assess the functional effects of ultrasound on neural activity, tFUS was used to modulate SSEPs in the rat somatosensory cortex elicited by electrical stimulation applied to the contralateral tibial nerve of the leg while systematically applying tFUS to the VPL nucleus of the thalamus. This sensory pathway extends through the thalamus, which is a good ultrasound target due to its central location where tFUS undergoes minimal distortion.

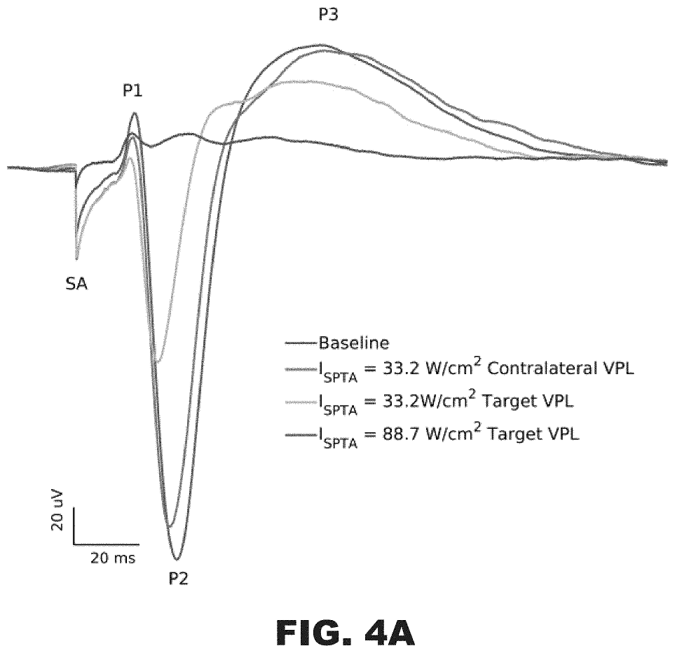

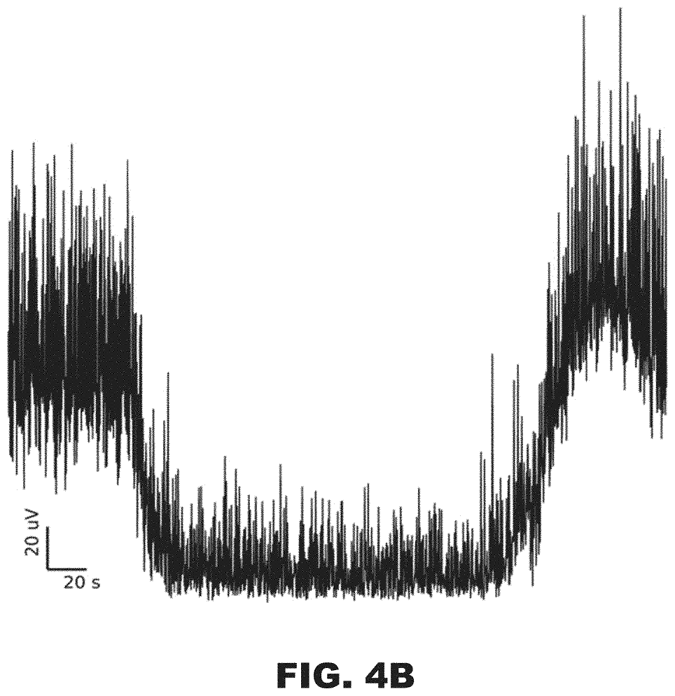

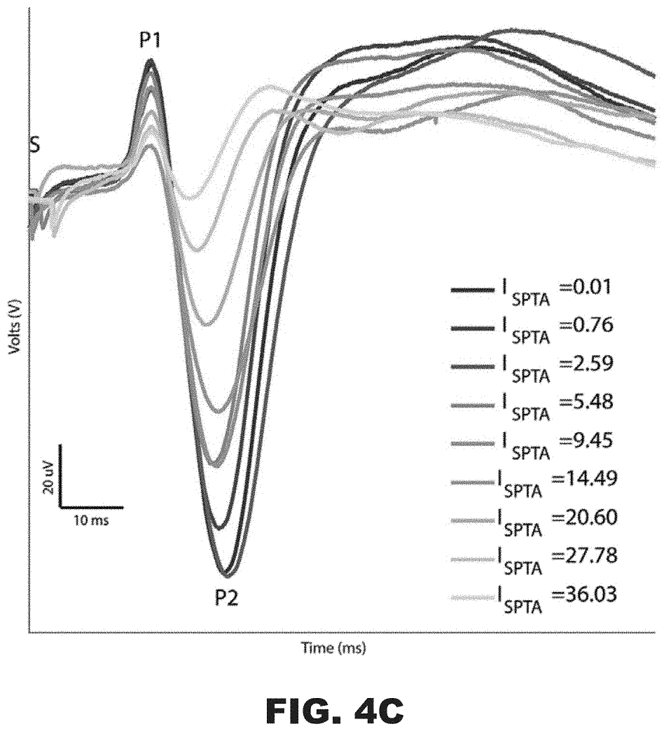

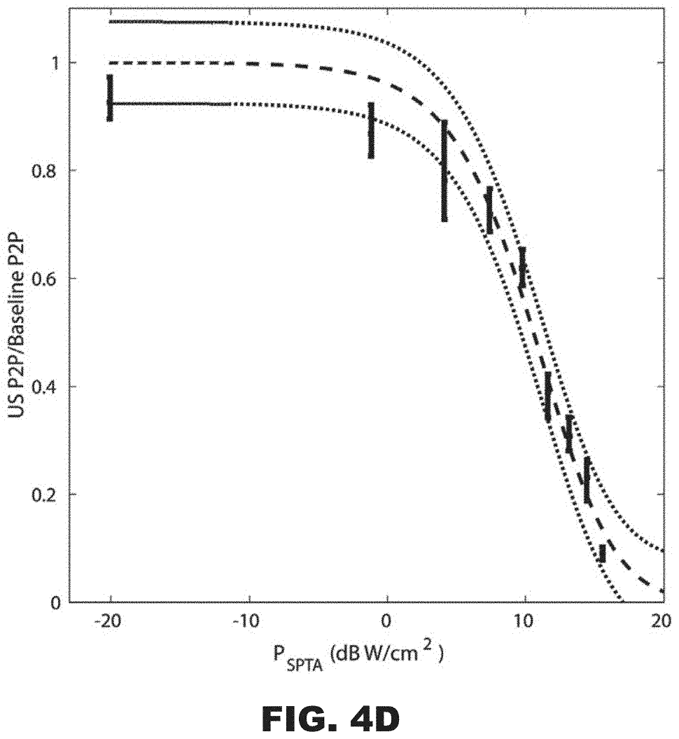

Rats were deeply anesthetized until they did not respond to toe pinch. No evoked or spontaneous movement was ever observed during the experiments or during ultrasound delivery. Tibial nerve stimulation produced large amplitude SSEPs measured epidurally over the contralateral hemisphere. Averaging 50 SSEP traces produced highly-resolved evoked potential waveforms, as depicted in FIGS. 4A and 4C, with the three dominant peaks. The first two peaks, P1 and P2, were very reliable across animals, while the third peak, P3, was variable and not present in all animals.

Ultrasound profoundly suppressed the SSEP waveform when focused on, or applied to, the contralateral VPL to stimulated tibial nerve, and at I_{SPTA}=88 W/cm² caused near-complete suppression (see FIG. 4A). Ultrasound focused on the opposite VPL nucleus, ipsilateral to tibial nerve stimulation, had no effect when compared to baseline. In general, the effects of VPL neuromodulation with ultrasound on average SSEP waveforms are shown in FIG. 4A. The effect of ultrasound was not immediate, taking about 20 seconds to achieve maximal effect. The effect of ultrasound on peak-to-peak amplitude of the SSEP over time, measured as the amplitude difference between the first and second peaks (P1 to P2), is shown in FIG. 4B. Recovery of the SSEP after cessation of ultrasound was complete after approximately 20 seconds. No ultrasound-evoked responses were observed and no changes in the power spectra were found.

Ultrasounds Intensity Determines SSEP Suppression

After establishing that FUS modulates the SSEP in a spatially specific and amplitude-dependent way, the effect of energy, or the relationship between delivered energy and suppression, was measured by systematically varying the ultrasound amplitude (peak pressure) on SSEP amplitude relative to baseline. Steady-state changes in the SSEP were measured. Suppression varied as a function of the I_{SPTA} as shown in FIG. 4C, which demonstrated the SSEP waveforms at different I_{SPTA} amplitudes, and the peak-to-peak amplitude of the SSEP as a function of the I_{SPTA}. The effect of SPTA power, P_{SPTA}, on SSEP suppression relative to baseline amplitude fit well with a sigmoid curve (FIG. 4D). Statistically significant differences between each power and every other power were found (p<0.0001) except between the smallest two. Specifically, at lower powers approaching zero, the peak-to-peak SSEP amplitude normalized to baseline, or the ratio of the peak-to-peak during ultrasound divided by the baseline peak-to-peak amplitude, approached 1. At higher powers, suppression of the SSEP saturated at around 15 dB W/cm². In general, all values above 5 dB had a statistically significant suppression of the SSEP compared to the baseline amplitudes, for example, as measured with a t-test.

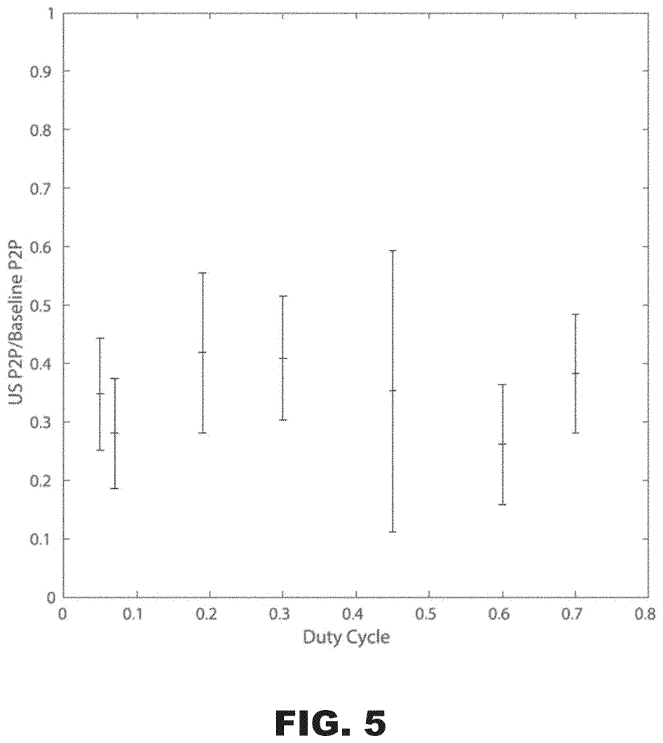

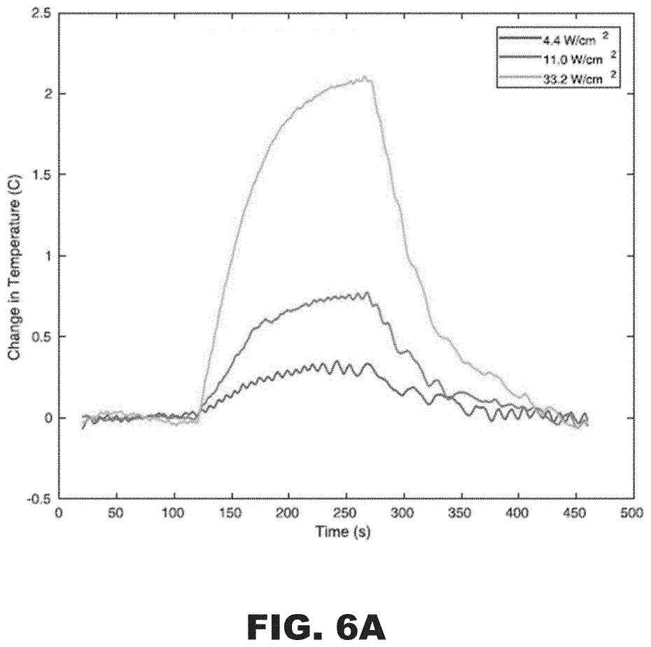

For pulsed, sinusoidal ultrasound waves, intensity varies with respect to the amplitude of the wave (peak pressures) and the duty cycle. To assess the effect of duty cycle on the SSEP suppression, ultrasound stimulation was delivered over a range of duty cycles while adjusting the amplitude to keep the I_{SPTA} at a constant mid-range 20 W/cm² intensity to assess the effect of duty cycle and amplitude on the SSEP suppression. The effect of FUS at a constant I_{SPTA} was relatively unchanged for duty cycles from 5% to 70% (FIG. 5). This energy level caused approximately 50% reduction in SSEP amplitude. No significant correlation between duty cycle (or the corresponding peak pressure) and SSEP normalized to baseline was found (R²<0.02). The lack of relationship between suppression and duty cycle when I_{SPTA} is held constant suggests a mechanism linked closely to the time averaged intensity rather than an independent effect from duty cycle, or even independent from peak pressure. Various modulation frequencies were also tested at two different intensities and found to have no significant effect on suppression.

Temporal Dynamics of Ultrasound Suppression of SSEP

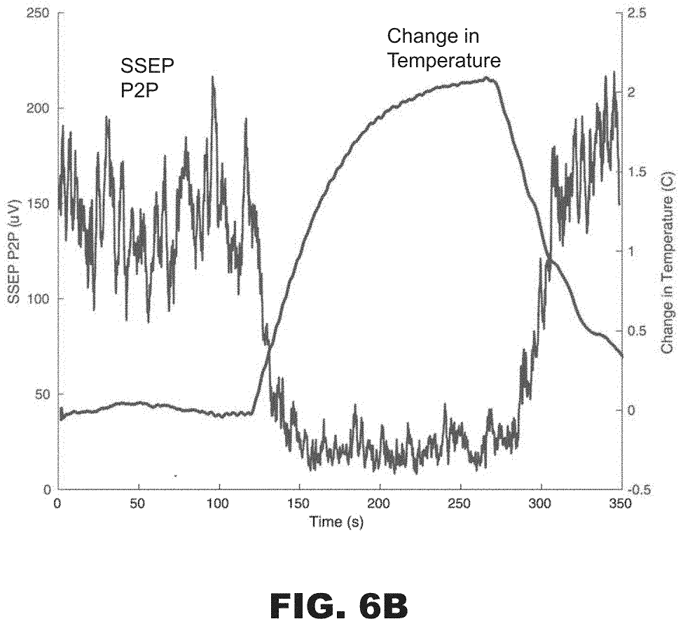

The effect of FUS on the suppression of the SSEP was not instantaneous, persisting for 10's of seconds beyond the ultrasound application, which is long for a neural mechanism. In view of the highly linear correlation between I_{SPTA} and in vivo temperature changes in the focus (see FIG. 3C), temperature changes were inferred during application of the ultrasound at three intensities shown in FIG. 6A (order from highest to lowest peak change in temperature: 33.2 W/cm², 11.0 W/cm², and 4.4 W/cm²). Compared to timescales typical for neural processes, the onset of the maximal effect and washout time were found to be long, on the order of 10's of seconds. A similar time scale was observed in the temporal dynamics of temperature change during heating and cooling, reinforcing that thermal heating is associated and may play a key role in the mechanism of suppression. The time course of SSEP suppression by tFUS was similar to that of the change in target temperature over time shown in FIG. 6B suggesting that temperature effects caused by ultrasound may be the primary mechanism of neuroinhibition. Superimposing the temperature change and SSEP peak-to-peak change, as shown in FIG. 6B, the temporal onset and washout with the temperature changes were able to be compared.

Temperature changes built up with a time constant on the order of 10's of seconds and dissipated with similar time constants. For these energy ranges, that can cause significant effects on SSEPs, the temperature effects were fairly linear. The suppression of the SSEP closely followed the temperature effects, suggesting that temperature may be a contributing mechanism for suppressing the SSEP.

Histology

A subset of animals underwent repeated treatment across a range of intensities and were allowed to recover. No animals showed chronic behavioral effects. After 3 to 7 days the animals were sacrificed, brains were extracted and underwent histological examination using Hematoxylin and Eosin (H&E) staining. Full sets of histology slides were reviewed by 3 qualified pathologists (H. Brent Clark, Director of Neuropathology; Flaviu Tabaran and Gerrald O'Sullivan from Comparative Pathology Shared Resource). No pathological effects of the delivered ultrasound were found.

### COMPARATIVE EXAMPLE

Neuromodulation with Laser Heating

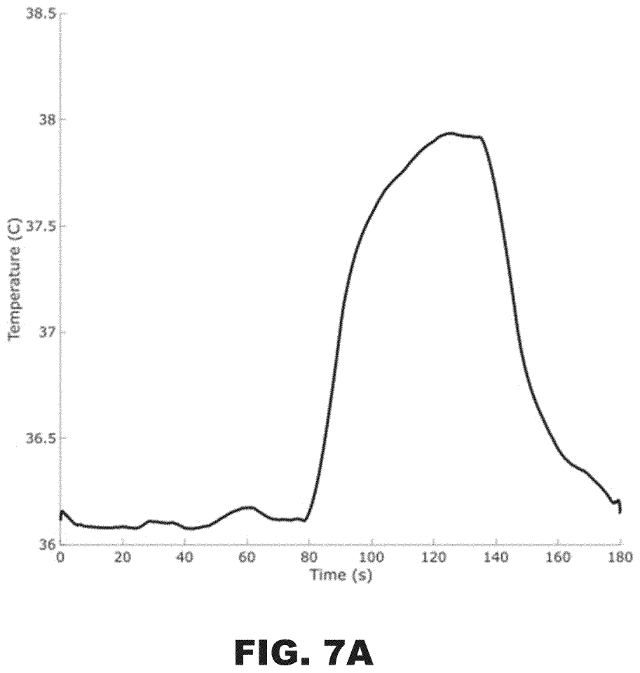

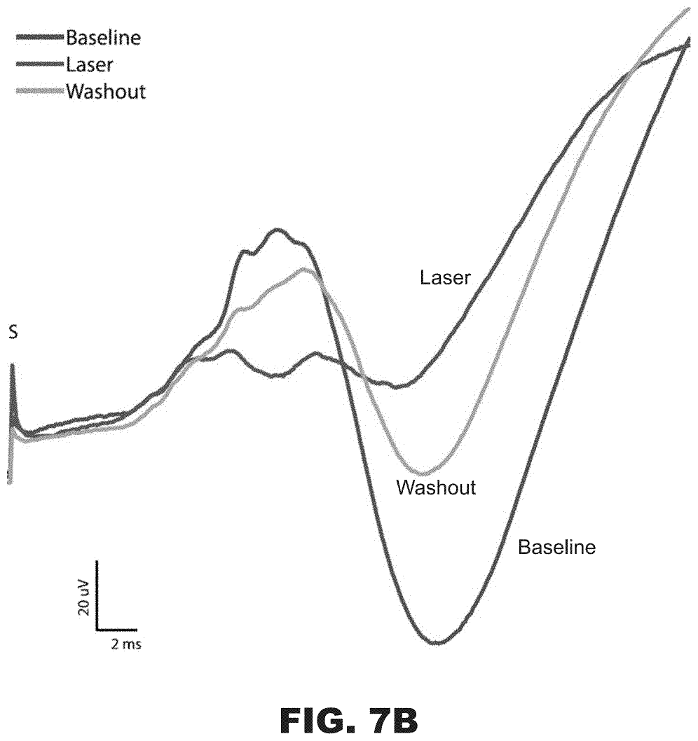

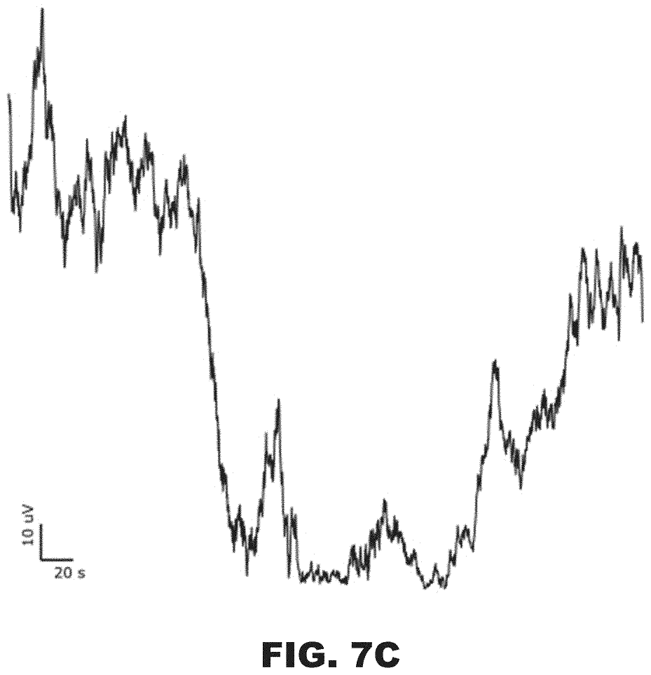

To isolate the effect of neuroinhibition by thermal energy, a laser was used to apply thermal energy through a fiber optic catheter to the VPL using the same SSEP experimental paradigm. To characterize the thermal effects of the laser, a fine-wire thermocouple probe placed under image guidance to allow direct assessment of the production of heat by the laser delivered through an optical fiber. The optical fiber was stereotactically placed along a vertical trajectory to the VPL where an optimal distance was experimentally obtained. Various distances below the cortex were evaluated to maximize the effect of the laser heating, as the maximal change in temperature is likely to be millimeters in front of the interface between the glass fiber and brain tissue. The red light laser had an absorptive volume comparable to that of tFUS. The time course of the temperature at the tip of the optical fiber in the brain is plotted in FIG. 7A. Similar to the ultrasound experiments, a 2° C. rise was observed and SSEP amplitude suppressed to about 70% of steady state within 20 seconds. The resulting average waveforms (FIG. 7B) revealed profound suppression of the peak-to-peak amplitude between P1 and P2, similar to that seen with the ultrasound (p<0.00003). The time course of suppression, as shown in FIG. 7C, was also similar to ultrasound suppression, supporting the hypothesis that the dominant effect of ultrasound is due to heating.

Despite an inherently different mechanism of heating, the effect on the SSEP was significant and similar to FUS, supporting that thermal energy may play a central role in focused ultrasound neuromodulation.

Thus, various embodiments of REVERSIBLE THERMAL NEUROMODULATION USING FOCUSED ULTRASOUND are disclosed. Although reference is made to the accompanying set of drawings that form part of this disclosure, one of at least ordinary skill in the art will appreciate that various adaptations and modifications of the embodiments described are within, or do not depart from, the scope of this disclosure. For example, aspects of the embodiments described may be combined in a variety of ways with each other. Therefore, it is to be understood that, within the scope of the appended claims, the claimed invention may be practiced other than as explicitly described.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as may refer to any of the foregoing structure or any other physical structure suitable for implementation of the described techniques. Also, the techniques could be fully implemented in one or more circuits or logic elements.

All scientific and technical terms used have meanings commonly used in the art unless otherwise specified. The definitions provided are to facilitate understanding of certain terms used frequently and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed or, for example, within typical ranges of experimental error.

The recitation of numerical ranges by endpoints includes all numbers subsumed within that range (e.g. 1 to 5 includes 1, 1.5, 2, 2.75, 3, 3.80, 4, and 5) and any range within that range. The terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a transducer array may be operatively coupled to a controller to deliver or receive electrical signals therebetween).

Terms related to orientation, such as “top,” “bottom,” “side,” and “end,” are used to describe relative positions of components and are not meant to limit the orientation of the embodiments contemplated. For example, an embodiment described as having a “top” and “bottom” also encompasses embodiments thereof rotated in various directions unless the content clearly dictates otherwise.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of” “consisting of,” and the like are subsumed in “comprising,” and the like.

The term “and/or” means one or all the listed elements or a combination of at least two of the listed elements.

The phrases “at least one of,” “comprises at least one of,” and “one or more of” followed by a list refers to any one of the items in the list and any combination of two or more items in the list.

All references and publications cited are expressly incorporated herein by reference in their entirety for all purposes, except to the extent any aspect directly contradicts this disclosure.

What is claimed is:

1. A method comprising:
positioning an ultrasound transducer system to direct focused ultrasound energy into a subject's tissue;
configuring the ultrasound transducer system to direct focused ultrasound energy into target nervous system tissue in the subject's tissue configured to cause heating of the target nervous system tissue to reversibly modulate neural activity; and
delivering focused ultrasound energy to target nervous system tissue based on the configured ultrasound transducer system.

2. The method of claim 1, wherein the ultrasound transducer system comprises a dual-mode ultrasound array configured to provide the focused ultrasound energy.

3. The method of claim 1, wherein the ultrasound transducer system is positioned outside of the subject's skull or outside of nervous system tissue.

4. The method of claim 1, wherein the focused ultrasound energy is configured to heat the target nervous system tissue at least 0.3 degrees Celsius at any location within the field of view of the focused ultrasound energy.

5. The method of claim 1, wherein the focused ultrasound energy is delivered for at least 1 second.

6. The method of claim 1, wherein I_{SPTA} of the delivered focused ultrasound energy at the target nervous system tissue is greater than 0.3 W/cm².

7. The method of claim 1, wherein I_{SPTA}of the delivered focused ultrasound energy is selected such that the target nervous system tissue is undamaged after delivering the focused ultrasound energy.

8. The method of claim 1, wherein the focused ultrasound energy has a duty cycle of up to 10%.

9. The method of claim 1, wherein the focused ultrasound energy has a carrier frequency of at least 1 MHz.

10. The method of claim 1, wherein the focused ultrasound energy has a focal spot size of less than 5 mm in at least two dimensions.

11. A system comprising:
a dual-mode ultrasound transducer configured to deliver focused ultrasound energy; and
a controller operably coupled to the dual-mode ultrasound transducer, the controller configured to drive the dual-mode ultrasound transducer to deliver focused ultrasound energy to cause heating of target nervous system tissue to reversibly modulate neural activity.

12. The system of claim 11, further comprising a dual-mode ultrasound array, comprising the dual-mode ultrasound transducer and at least one other dual-mode ultrasound transducer, to provide the focused ultrasound energy.

13. The system of claim 11, wherein the dual-mode ultrasound transducer is positioned outside of the subject's skull or in at least partially outside of nervous system tissue.

14. The system of claim 11, wherein the focused ultrasound energy is configured to heat the target nervous system tissue at least 0.3 degrees Celsius at any location within the field of view of the focused ultrasound energy.

15. The system of claim 11, wherein the focused ultrasound energy is delivered for at least 1 second.

16. The system of claim 11, wherein I_{SPTA} of the delivered focused ultrasound energy at the target nervous system tissue is greater than 0.3 W/cm².

17. The system of claim 11, wherein I_{SPTA} of the delivered focused ultrasound energy is determined such that the target nervous system tissue is undamaged after delivering the focused ultrasound energy.

18. The system of claim 11, wherein the focused ultrasound energy has a duty cycle of up to 10%.

19. The system of claim 11, wherein the focused ultrasound energy has a carrier frequency of at least 1 MHz.

20. The system of claim 11, wherein the focused ultrasound energy has a focal spot size of less than 5 mm in at least two dimensions.
